(19) United States
(12) Patent Application Publication
(10) Pub. No.: US 2024/0386995 A1
(43) Pub. Date: Nov. 21, 2024

(54) Systems and Methods for Detecting CRISPR-Mediated Residues Within Methylated Patterns of Genome Using Automated Statistical Methods and Long Short-Term Memory Autoencoders

(71) Applicant: MITRE Corporation, McLean, VA (US)

(72) Inventors: Heath Farris, McLean, VA (US); Patrick Hinson, McLean, VA (US); Tyrone V. Patterson, III, McLean, VA (US); Eliza M. Mace, McLean, VA (US); Pamela A. Texter, McLean, VA (US); Kevin R. Gemp, McLean, VA (US); Sybil A. Russell, McLean, VA (US)

(21) Appl. No.: 18/199,801

(22) Filed: May 19, 2023

(51) Int. Cl. G16B 20/20; G06N 3/0442; G06N 3/0455; G06N 3/0464; G16B 40/20

(52) U.S. Cl. CPC G16B 20/20; G06N 3/0442; G06N 3/0455; G06N 3/0464; G16B 40/20

(57) ABSTRACT

A system, method, and computer-readable medium for detecting a CRISPR-edited genome are disclosed. Certain embodiments of the system may include one or more processors configured to receive input sequence data of a genome; provide the input sequence data to a long short-term memory (LSTM) autoencoder neural network (ANN) having at least one encoder layer and at least one decoder layer, wherein the LSTM ANN was trained using a training data sequence of a genome without CRISPR edits; reduce, using the encoder layer, a dimensionality of the input sequence data to generate reduced data; restore, using the decoder layer, a dimensionality of the reduced data to generate restored data; statistically compare the input sequence data and the restored data to identify anomalies in the genome; and determine, based on a result of the statistical comparison, whether the genome contains a CRISPR-edited methylation region. A corresponding method and computer-readable medium are also provided.

### TECHNICAL FIELD

The present application pertains to systems and methods for detecting CRISPR-mediated alterations to methylome regions using statistical methodology and/or long short-term memory autoencoder neural networks and training such networks. More specifically, the present application pertains to systems and methods for detecting CRISPR-mediated alterations to cytosine-phosphate-guanine (CpG) island (CGI) and sub-CGI locations using automated statistical methods and/or long short-term memory autoencoders and training such autoencoders.

### BACKGROUND

A number of gene editing methods exist that provide means to treat genetic, viral, and bacterial diseases. In particular, CRISPR (Clustered Regularly Interspaced Short Palindromic Repeats) gene editing is a powerful tool for generating genomic edits with high precision and efficiency. CRISPR involves creating a DNA double-strand break (“DSB”) at a target site. Following the creation of the DSB, the cell containing the DNA may use one of several processes, such as non-homologous end joining (NHEJ), to repair the DSB. During NHEJ, nucleotides may be added or removed by the cell, which results in a sequence that is different from the original targeted sequence. Alternatively, the cell may repair a DSB by homology-directed repair (“HDR”) or homologous recombination (“HR”) mechanisms, which utilize an endogenous or exogenous donor template with homology to each end of the DSB to direct repair of the break.

The vertebrate genome consists of regions comprised of a high number of CpG dinucleotides, which are known as CpG islands (“CGIs”). In mammals, CGIs are targets of methylation, and the methylation patterns across the genome are reset and reestablished during embryogenesis. CGIs are typically located in gene regulatory elements, such as promoters and enhancers. The methylation of CGIs plays a role in whether a gene is active or inactive.

Multiple factors can influence the methylation state of the genome. For example, studies have shown that CRISPR can alter the methylation patterns of CGIs within the region of the genome that is being targeted. Specifically, when CRISPR targets a CGI region, the CRISPR-generated edits can result in an increase in methylation of the CGIs. Thus, there exists a need for the development of a method for detecting changes in methylation as a result of CRISPR-mediated genome editing.

### SUMMARY

According to certain embodiments, a system for detecting a CRISPR-edited genome is disclosed. The system includes one or more processors configured to receive input sequence data of a genome. The input sequence data is provided to a long short-term memory autoencoder neural network (LSTM) having at least one encoder layer and at least one decoder layer. The LSTM was trained using a training data sequence of a genome without CRISPR edits. Using the encoder layer, a dimensionality of the input sequence data is reduced to generate reduced data. Using the decoder layer, a dimensionality of the reduced data is restored to generate restored data. The processors statistically compare the input sequence data and the restored data to identify anomalies in the genome and determine, based on a result of the statistical comparison, whether the genome contains a CRISPR-edited methylation region.

According to certain embodiments, a method for detecting a CRISPR-edited genome is disclosed. The method includes receiving input sequence data of a genome. The input sequence data is provided to an LSTM having at least one encoder layer and at least one decoder layer, wherein the LSTM was trained using a training data sequence of a genome without CRISPR edits. Using the encoder layer, a dimensionality of the input sequence data is reduced to generate reduced data. Using the decoder layer, a dimensionality of the reduced data is restored to generate restored data; statistically comparing the input sequence data and the restored data to identify anomalies in the genome. Then, based on a result of the statistical comparison, the method determines whether the genome contains a CRISPR-edited methylation region.

According to certain embodiments, the present disclosure describes a non-transitory computer-readable storage medium storing instructions that when executed by a computer cause the computer to perform a method for detecting a CRISPR-edited genome. The method is characterized by receiving input sequence data of a genome. The input sequence data is provided to an LSTM having at least one encoder layer and at least one decoder layer, wherein the LSTM was trained using a training data sequence of a genome without CRISPR edits. Using the encoder layer, a dimensionality of the input sequence data is reduced to generate reduced data. Using the decoder layer, a dimensionality of the reduced data is restored to generate restored data. The input sequence data and the restored data are statistically compared to identify anomalies in the genome and based on a result of the statistical comparison, the method determines whether the genome contains a CRISPR-edited methylation region.

### BRIEF DESCRIPTION OF THE DRAWINGS

Embodiments of the present disclosure will be described with reference to the accompanying drawings, in which:

FIG. 1 is a flow diagram of a process for detecting CRISPR-mediated methylome residues using a long short-term memory (LSTM) autoencoder in conjunction with statistical methodologies, in accordance with some embodiments.

FIG. 2 is a flow diagram of a process for training an LSTM network to detect CRISPR-mediated methylome residues in conjunction with statistical methodologies, in accordance with some embodiments.

FIG. 3 is a machine-learning process diagram for the LSTM autoencoder.

FIG. 4 provides an example of the statistical process to compare input data with the values predicted by the LSTM autoencoder.

FIG. 5 depicts an example of a statistical filtration process to categorize and rank CGI methylation patterns while filtering out subsets of data that fail to meet thresholds for significance.

FIG. 6 is flow diagram of another process for detecting CRISPR-mediated methylome residues using a trained LSTM network and a second neural network, in accordance with some embodiments.

FIG. 7 illustrates an example user interface for indicating that a genome contains one or more methylation regions that has been CRISPR-edited.

### DETAILED DESCRIPTION

The field of epigenetics pertains to the study of changes to a genome that do not involve changes to its nucleotide sequence and their phenotypic effects. DNA methylation and demethylation are the primary mechanisms involved in conferring epigenetic change and therefore heavily control the expression of all genes that comprise a genome. The expression of physical traits in animals is highly dependent on the regional levels of methylation enrichment occurring at the genes that encode for those traits. Currently, there is no dedicated, reliable, universal tool available with which to detect and track changes to methylation patterns incurred during the use of CRISPR technology to edit a genome. This underscores an inherent risk and potential shortcoming in the mass adoption of CRISPR techniques—an inability to anticipate the actual, phenotypic outcome of a particular edit.

Cytosine-phosphate-guanine (CpG) sites are regions of DNA where a cytosine nucleotide is followed by a guanine nucleotide in a linear sequence of bases and are often targets of important epigenetic activity. CpG sites occur with high frequency in genomic regions called CpG islands (CGIs). CGIs are typically located in gene promoter regions, gene enhancer regions, or within genes themselves and may play an important role in the biological regulation of gene expression. CGIs are commonly maintained in a hypomethylated state. Inducing changes to these preserved methylation patterns within the epigenome can have transgenerational effects, with progeny exhibiting the same modifications created within the parental strains. Unintended deregulation of regulatory elements due to the collateral effects of CRISPR editing could likely have unpredictable consequences for the cell and hence the organism. In particular, CRISPR edits leveraging homology directed repair (HDR) mechanisms in combination with donor homology arms localized around CGIs (and even sub-CGI CpG sites) induce modifications of the methylation patterns occurring at these genomic sections, resulting in distinctively augmented and persistent methylation within the recombinant region.

The following provides a disclosure of systems and methods for detecting genomic scars inflicted by application of CRISPR (i.e., in which the original methylation states of cytosine bases become permanently reversed). Additionally, the disclosed embodiments combine statistical analysis with algorithmic sorting to identify genomic scars with precision (i.e., pinpointing a particular CGI region or sub-CGI location). The detection process is applicable to a wide-range of organisms. Across different mammalian systems, CpG locations are available for targeted methylation, and many species have been genetically modified using CRISPR and CRISPR-derived technologies. Additionally, epigenetic residues imparted by CRISPR-mediated incorporation of donor DNA using HDR mechanisms are conserved among mammalian systems, providing further support for the broad application of this detection methodology. Mammalian applications include human cell cultures, non-human primates, rats, elephants, goats, pigs, mice, and cows.

The detection process involves training a long short-term memory (LSTM) autoencoder to recognize patterns occurring within raw whole genome bisulfite sequencing (WGBS) data. The disclosed embodiments allow for fast, precise, and reliable examination and diagnosis of a CRISPR-modified genomic sample by pinpointing an affected particular loci, CpG site(s), and/or CGI. Long short-term memory (LSTM) autoencoders are a type of neural network that can process and predict sequences of data. LSTM autoencoders are trained to reconstruct a sequence by learning to predict the next step in the sequence, given the previous steps. They are called “autoencoders” because they can automatically learn to compress and reconstruct their input data. LSTM autoencoders can be used for anomaly detection by learning to reconstruct normal data and then flagging input data that cannot be accurately reconstructed as anomalous. The long-term memory component of the network enables the autoencoder to remember important information from the input sequence for a longer period of time, allowing it to better process long-term dependencies in the data.

LSTM autoencoders are composed of two main components: an encoder and a decoder. The encoder part of the neural network reduces the dimensionality of the input data by generating a compact representation of the input data. Dimensionality refers to the number of variables and/or attributes recorded by a given dataset. If the dataset were to be organized into a two-dimensional array of rows and columns, then the dimensionality would be analogous to the number of rows and columns. The encoder typically consists of a series of layers (or gates) that selectively let data pass through to the next layer. Within each layer of the encoder, the input data is partially discarded (or “forgotten”), reducing the dimensionality of the input data as it is passed through the neural network. The amount of data to be let through a given layer of the encoder is determined by a sigmoid function which outputs numbers between zero and one. A value of zero corresponds to completely discarding all data prior to entering the next layer, while a value of one corresponds to passing all data along to the next layer (thereby retaining all input data).

Dimensionality reduction is a technique for reducing the size of the data in a dataset in order to facilitate analysis of the dataset. Reducing the dimensionality involves removing extraneous data or features from a dataset so as to preserve only the most important information. It is often used when working with high-dimensional datasets, as they can be difficult to visualize and analyze due to the large number of features. For example, information stored within an array comprising the dimensions of 16×16×2 may be compressed (or “bottlenecked”) to an array comprising the dimensions of 8×8×2. The data may be subjected to multiple rounds of dimensionality reduction (i.e., passed through multiple layers of the encoder). Dimensionality reduction can make it easier to understand the relationships between the features and the target variable, and can also help to reduce the computational cost of working with large datasets.

There are several techniques for dimensionality reduction, including principal component analysis (PCA), linear discriminant analysis (LDA), and t-distributed stochastic neighbor embedding (t-SNE). These techniques transform the data into a lower-dimensional space, where the dimensions are chosen to capture the most important information from the original dataset.

The decoder receives the dimensionally reduced dataset (which preserves the intrinsic features of the input sequence data) and produces a reconstructed sequence that approximates the original input sequence. Reconstructed sequences need not be identical to their original counterparts; rather, reconstructed sequences are intended to emulate patterns that occur throughout the input sequence data within a predefined accuracy threshold. The performance of the decoder is evaluated based on its ability to recreate the input sequence.

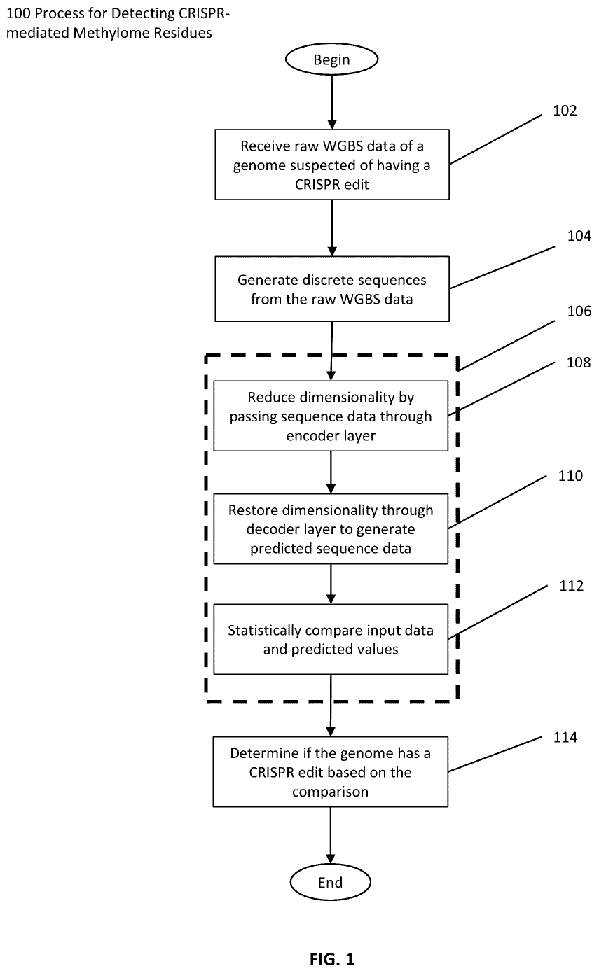

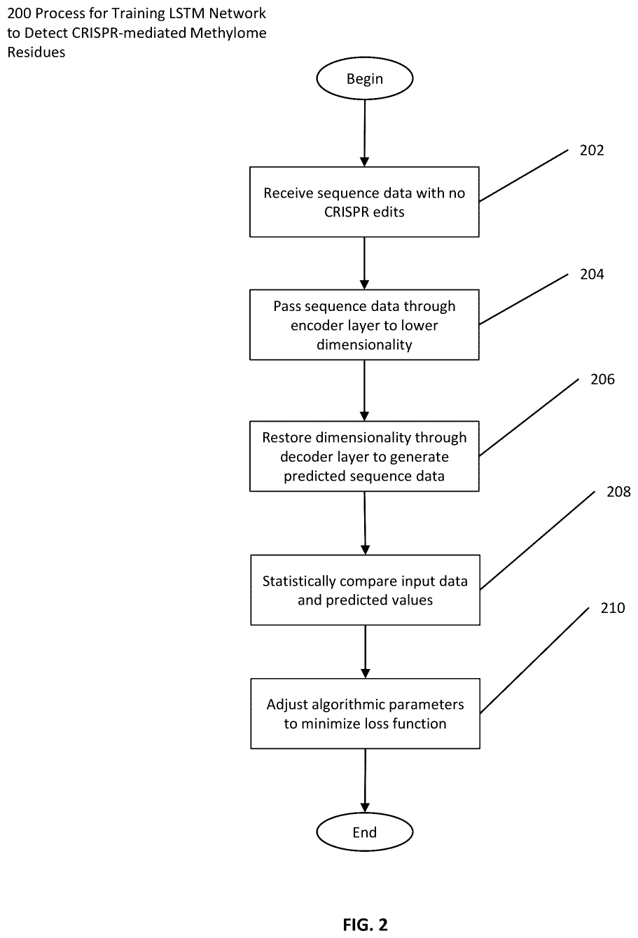

FIG. 1 illustrates a flow diagram of a process 100 for detecting CRISPR-mediated methylome residues using an LSTM autoencoder, in accordance with some embodiments. In the example of FIG. 1, process 100 may further use statistical methodologies (not shown in FIG. 1) to automate the search and identification of stable epigenetic scarring of a genome produced by CRISPR-editing activity. In FIG. 1, before process 100 is executed, the LSTM has been previously trained to a degree such that the LSTM is capable of identifying a CRISPR-edited region of a genome to a predetermined level of accuracy. The LSTM may have been trained, for example, using process 200 described below with respect to FIG. 2.

Prior to step 102, sequence data may be derived from various tissue samples of organisms of interest. In some embodiments, these samples may be tissue-specific or they may comprise whole embryo samples. The genomic DNA may be then extracted and purified from these tissue samples. In embodiments wherein the sequence data is whole-genome bisulfite sequencing (WGBS) data, the genomic DNA receives a bisulfite treatment that converts each unmethylated cytosine nucleotide within the DNA to a distinguishable uracil nucleotide while leaving their methylated counterparts unaffected. Thus, the detection of any remaining cytosine residues amounts to the detection of methylation at those bases.

In some embodiments, various genomic sites of interest may be enriched prior to or during sequencing using, for example, restriction enzymes or immunoprecipitation.

Following bisulfite treatment of whole genome sample extractions, next-generation DNA sequencing (NGS) is used to sequence and assemble the entire length of the genome (within which each methylated cytosine will be detected and mapped). NGS leverages massively parallel processing technology to simultaneously sequence redundant fragments of DNA that variously map to different regions of the genome and together cover its entire length multiple times over. These sequenced fragments are then read, aligned, and assembled on the basis of their overlapping areas. The accuracy of this method improves as the number of genomic copies that are sequenced and overlapped for comparison increases. As such, depth of coverage in DNA sequencing is quantified as the amount of overlap and/or congruence that is detected during the alignment stage. For example, if a cytosine occurring at a specific genomic location is detected five (5) times (across five sequence fragments overlapping at that base), that base has a depth of five (5). Given the variability associated with genomic methylation patterns, which may vary substantially despite being sourced from the same tissue sample, methylation coverage for a given cytosine residue may be presented as the percentage of cytosines detected out of all nucleotides (cytosines+converted uracils) detected at that same location (which should also be equivalent to the depth of coverage for the entire genome sequenced).

For example, if, out of ten complete, NGS-sequenced and aligned genomic copies, five copies call the methylation of a cytosine at a particular CpG site, then the methylation rate for that particular site would be 50%. If a CRISPR-edited, genomic counterpart reported a methylation rate of 75% at that same CpG site, then the methylation difference between the baseline and edited sites would be +25%. Furthermore, if a CRISPR-edited, genomic counterpart reported a methylation rate of 25% at that same CpG site, then the methylation difference between the baseline and edited sites would be −25%. The methylome information encoded in the sequence data may be obtained by sequencing across the genome at a sufficient depth of coverage to capture all high-density CpG regions of interest.

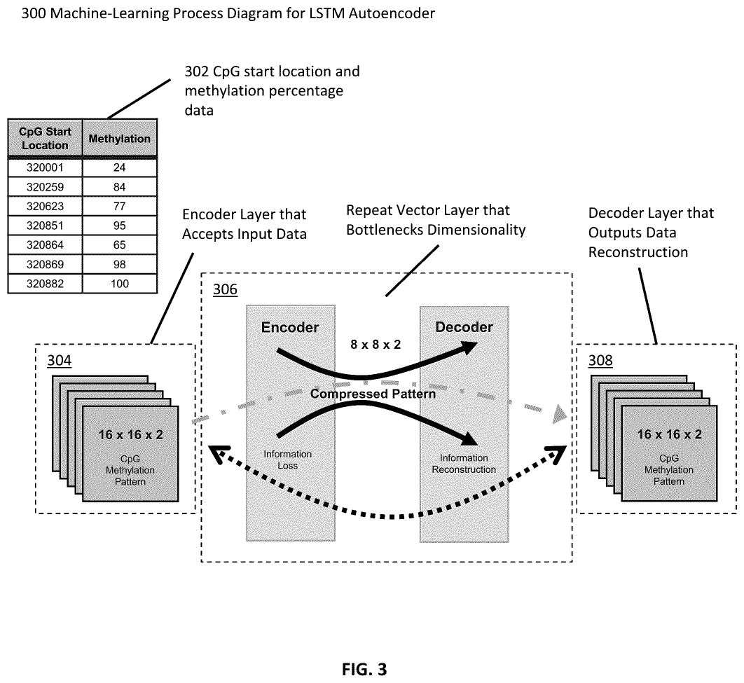

The above approach may be used after sequencing during a step 104 in order to quantify the relative amounts of methylation within a genomic sequence. Table 302 of FIG. 3 additionally illustrates a set of data sequences that reflects the variance among methylation levels throughout a given chromosome. The generation of these sequences is elaborated upon in the description of step 104 of FIG. 1.

At step 102, a processor receives sequence data of a genome suspected of having one or more CRISPR-edited methylation regions. In some embodiments, the sequence data may be received from a gene sequencing machine (e.g., via local network or the internet). Alternatively, or additionally, the sequence data may be provided by a user (e.g., using a USB drive).

In a step 104, a processor generates discrete sequences from the raw WGBS data. Specifically, raw WGBS data is converted to a .bedGraph file format containing data of CpG locations and their methylation values for each chromosome in the entire genome of the sample. The .bedGraph files are converted to matrices containing consecutive sequences of CpG start locations and their methylation percentage values. These matrices can then be fed into the neural network that performs the steps included by the dotted box 106 of FIG. 1. Within the .bedGraph file, methylation percentage is calculated by dividing the number of methylated bases observed at that location by the total number of methylated and unmethylated bases observed at that location and multiplying by 100.

In some embodiments, statistical normalization or standardization techniques (such as min-max scaling) may be applied to data points when defining sequences across the genome to improve the overall performance and/or efficiency of the neural network. In further embodiments, other genomic data can be read in from the .bedGraph files and incorporated into the matrices that are fed into the neural network. The incorporation of genomic sequence data into the matrices, for example, can enable the parallel detection of inserted edits and modifications or deletions of protospacer adjacent motifs (PAMs).

The group of steps indicated by the dotted box 106 includes steps performed by an LSTM autoencoder. The LSTM autoencoder may be implemented on the same or different processor that performs steps 102 and/or 104. Alternatively, or additionally, the LSTM autoencoder may be implemented on a remote device or a remote cloud system (e.g., Google Cloud). The sequence data generated at step 104 is presented as an input to the LSTM network, which may incorporate one or more encoder layers and one or more decoder layers. In the example of FIG. 1, the LSTM network includes one encoder layer that performs step 108 and one decoder layer that performs step 110.

At step 108, the sequence data generated at step 104 is passed into an encoder layer, which reduces the dimensionality of the data. The dimensionality is lowered by passing the data through additional encoder layers so that it contains less information than the original input sequence. Specifically, the encoder has been trained to strip away extraneous, obfuscating, or otherwise non-essential datapoints (i.e., “noise”) while preserving datapoints that are deemed sufficiently valuable, which will later serve as a foundation upon which the decoder will build back a replica of the input sequence. The value judgement by the encoder is based on the training regimen that the network was previously subjected to, which exclusively involved emulating “normal” sequence data (i.e., lacking any CRISPR scars). The encoder is thus adapted to discard values that deviate from the archetypal behavior of normal methylation sequences, which includes datapoints that reflect anomalous patterns indicative of CRISPR modification. The reduced output from the encoder is then provided to a decoder.

At step 110, the decoder attempts to reconstruct the original input from step 102 through an inverted mechanism. The original dimensionality of the data is gradually restored as the data progresses through successive decoder layers. For example, information originally stored within an array comprising the dimensions of 16×16×2 may be compressed to an array comprising the dimensions of 8×8×2. A decoder will restore the dimensionality of this dataset to 16×16×2 by applying the sequence-patterns that were learned during training to predict the missing values in the compressed dataset. By accurately interpolating datapoints that are consistent with non-CRISPR edited sequences, the decoder will thus generate a reconstructed sequence that conforms closely to normal sequences but deviates significantly from the regions of an input sequence that contain a CRISPR-edit. This subsequent “error” (i.e., the magnitude by which the value predicted by the decoder failed to match its original counterpart) serves as an indication that a CRISPR-edit is present within those regions of the input sequence.

The encoder and decoder are each composed of layers of LSTM units. For example, an encoder and decoder may each comprise of two LSTM layers, the encoder's first being composed of 64 LSTM units and the encoder's second being composed of 48 units, and the decoder's first layer being composed of 48 units, and the decoder's second layer being composed of 64 units. The data passes through each layer as it passes through the overall network. Therefore, in this example, the data will first pass through the 64 units of encoder's first layer, be manipulated by the weights in that layer, and then the data with newly manipulated values is passed through the 48 units of the encoder's second layer, and so on.

Mean Absolute Error (MAE) is a measure of the average magnitude of the errors in a prediction model and is commonly used as a metric for evaluating the performance of regression models. It is calculated as the sum of the absolute differences between the predicted values and the actual values, divided by the number of predictions.

MAE can be used to compare the performance of different models, or to tune the parameters of a model to achieve the best performance.

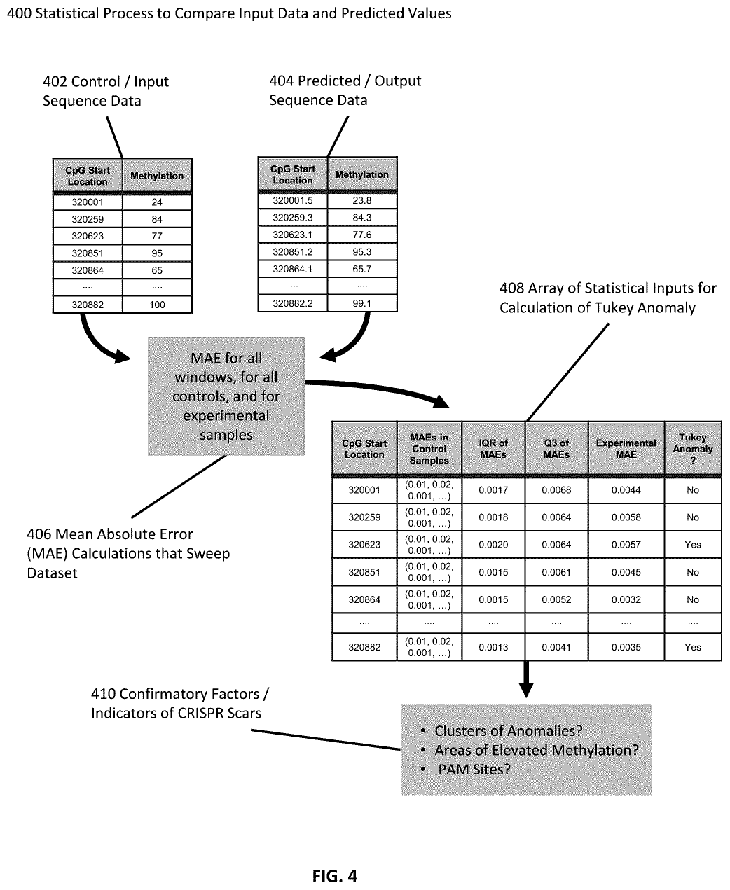

Restoration of dimensionality occurring in step 110 requires the model to make predictions as to the identity (in this case, methylation percentages and/or CpG locations) of the sequence elements that were removed by the encoder. The trained network is able to accurately identify and fill-in blank CpG locations and/or unknown methylation percentages that fall within the parameters of standard biological noise and natural variance. However, the effect of CRISPR-editing on the methylome distorts these values, and these distortions may be detected using statistical methods. As such, at a step 112, the model quantitatively evaluates whether a significant departure from the predicted sequence values for a particular dataset have occurred by way of a statistical comparison. This process is detailed by FIG. 4 (described below). At step 114, the model determines whether a CRISPR edit is present based on the result(s) of statistical tests performed in step 112.

FIG. 2 is a flow diagram of an example process 200 for training the LSTM network used for detecting CRISPR-mediated methylome residues in conjunction with statistical methodologies, in accordance with some embodiments. Specifically, FIG. 2 illustrates the training process used to reconstruct methylation calls at specific loci from a set of methylation sequence data derived from control samples, thus enabling the detection of anomalous locations (i.e., CRISPR-edited) based on the identification of statistically significant outliers. In general, the supervised training process enables the LSTM autoencoder to predict methylation sites and variance within a normalized threshold.

Process 200, which closely resembles process 100, may be used as an initial training process for the LSTM prior to its utilization within process 100. Once the LSTM has reached a satisfactory level of performance (i.e., is able to reliably detect CRISPR residues within new, unseen methylation datasets with a high degree of accuracy) following an extended period of training and validation, the mature, trained LSTM may be considered competent to perform the group of steps 104 (and thus eligible to participate in process 100). Alternatively, or additionally, process 200 may be used to further train the LSTM that has already been trained to produce results at or better than the predetermined level of accuracy. For example, after using process 100 of FIG. 1 to determine whether sequence data of a genome has a CRISPR edit (and/or the methylation location of such an edit), the sequence data may be fed back into the LSTM as the sequence data of step 202 in process 200 thereby further training the LSTM.

In order for a given artificial neural network (ANN) to provide accurate screening, diagnosis, or perform comparisons in general, it must be trained using a foundational data set that is derived from or is representative of its intended population of interest. In this case, the LSTM autoencoder may be trained to define normative methylation sequence structure using an appropriately large control library, followed by flagging anomalies detected after supplying CRISPR-edited methylome data. In the example of FIG. 2, in a step 202, training data is comprised of unedited sequence data (i.e., containing no CRISPR-edits) so that the model can learn the patterns observed in data that are representative of biological noise and natural variance. This establishes a baseline of expected and/or ground-truth sequence patterns such that when CRISPR-edited sequences are fed into the model, they are distinguishable as sources of generalization error during prediction attempts and are therefore classifiable as anomalous. The series of steps following step 202 in FIG. 2 conforms closely to the procedure encompassed by process 100 in FIG. 1.

In addition to training on unedited sequence data, synthetic (i.e., contrived) datasets comprising slightly modified copies of preexisting data can be used to supplement the LSTM network. The number of samples can be augmented by ‘spiking’ sequences with outlier-values or inserting data to artificially replicate a variety of edit-types (i.e., full vs. partial edits and variance of observed methylation frequencies). In some embodiments, these approaches may be used to test the limits of detection of the algorithm and calibrate its sensitivity, as well as expand a limited pool of sequence samples on which to train or to combat an overfitted model.

In a step 204, sequence data from step 202 is passed through an encoder layer to lower its dimensionality. Step 204 is similar to the step 106 of process 100 in FIG. 1. Likewise, step 206 and step 108 of process 100 both comprise of restoring dimensionally by way of a decoder layer that is used to predict and backfill missing sequence data. In step 208, the difference between the input data and predicted values are measured. However, in contrast to detection process 100, prediction accuracy is improved within process 200 by minimizing the MAE at step 210.

Some embodiments of LSTM networks may utilize a parameter optimization framework, allowing for the systematic and automatic optimization of training variables. This helps to significantly reduce the down-time of training cycles by improving the training efficiency of the network. Hyperparameters such as learning rate (measure of how rapidly the model moves to the minimum achievable error), dropout (the factor that randomly negates LSTM cells during model training step to combat overfitting data), number of LSTM units per layer, and number of LSTM layers may be chosen and fine-tuned.

In some embodiments, the LSTM process utilizes an Optuna parameter optimization framework, which allows for systematic and automatic optimization of training variables, which in turn, reduces the down-time of training cycles.

Hyperparameters such as the number of layers in the encoder and/or decoder (1, 2, or 3), the number of units per layer (16, 24, 48, 64, 128, or 256), the amount of dropout per layer (0.001, 0.005, 0.01, 0.05, or 0.1), choice of optimizer (ADAM, SGD, or SGD with Momentum), and learning rate (0.001, 0.005, 0.01, 0.05, or 0.1) may be selected.

FIG. 3 is a machine-learning process diagram for the LSTM autoencoder 300. LSTM autoencoders are particularly useful for processing sequential data, such as time series, natural language, and audio. They work by reading input data one time step at a time and using the previous time steps to predict the next time step.

In a step 304, the sequence data from table 302 is provided to the encoder of the LSTM network, where weights are initialized randomly and adjusted throughout the training epoch to minimize reconstruction loss. Weights are the coefficients that are associated with each LSTM node in the model and their connections. Specifically, the weights are initialized randomly by the model building software, TensorFlow, dictated by a random seed selected at the start of model training.

In a step 306, the encoder forces information loss on the data from step 304, reducing its dimensionality. In the example of FIG. 3, the data dimensionality is reduced from 16×16×2 to 8×8×2. Then, in a step 308, a decoder attempts to reconstruct the initial input based on the data generated at step 306, restoring the original dimensionality (in this case, 16×16×2). A RepeatVector layer (as opposed to an aforementioned LSTM layer) is a type of layer composed of vectors that replicate the values that are input to it and acts as the bridge between the encoder and the decoder. The RepeatVector layer is enclosed within step 306. A TimeDistributed layer handles converting the decoder output back to a sequential form as part of step 308. MAE is used for the loss function of the model.

FIG. 4 provides an example of a statistical process 400 to compare input data with the values predicted by the LSTM autoencoder. Process 400 performs steps similar to step 110 of process 100 and step 208 of process 200.

CRISPR-mediated genomic edits are identified by their high variance relative to normal methylation noise expected to occur naturally within the epigenome. However, epigenome regions generally do not conform to a normal distribution of reconstruction loss (i.e., how close the data output is to the original input). Therefore, in accordance with some embodiments, a Tukey test may be applied to the output data to reveal anomalous regions within the sample epigenomes. A Tukey test is a statistical test used to determine if there are significant differences between the means of two or more groups. Its calculation is described in detail below. The example in FIG. 4 defines anomalies as those CpG windows that score above the 3rd quartile values by a margin determined by a Tukey constant multiplied by the Interquartile range.

In process 400, original input and/or control data 402 is passed through the pretrained model corresponding to process 100 of FIG. 1 and predicted sequence data 404 is generated as a result. Calculations of MAE 406 are then performed for the output sequences across the sample set. An array 408 of the 75th percentiles (Q3) of the MAEs and the interquartile ranges (IQR) for each CpG location is generated. The MAEs from the experimental sample predictions are then combined with the Q3 and IQR values to determine whether the methylation pattern of a given CpG location constitutes a Tukey anomaly within the experimental sample.

Locations where CRISPR-mediated edits have been performed within the genome are likely to have many Tukey anomalies. In some embodiments, a clustering script may be used to identify regions within the genome where there is an accumulation of anomies.

The application of CRISPR technology for genomic edits often requires the destruction of protospacer adjacent motif (PAM) sites located near the CRISPR target location. In some embodiments, this association may be leveraged to corroborate positive identifications by the neural network. As such, in the example of FIG. 4, once statistical anomalies have been identified within the genomic region of interest, co-occurring indicators of CRISPR-mediated genomic editing 410, including observations of disrupted PAM sites and/or increased methylated patterns of the targeted genomic region, may be used to corroborate CRISPR-scar predictions.

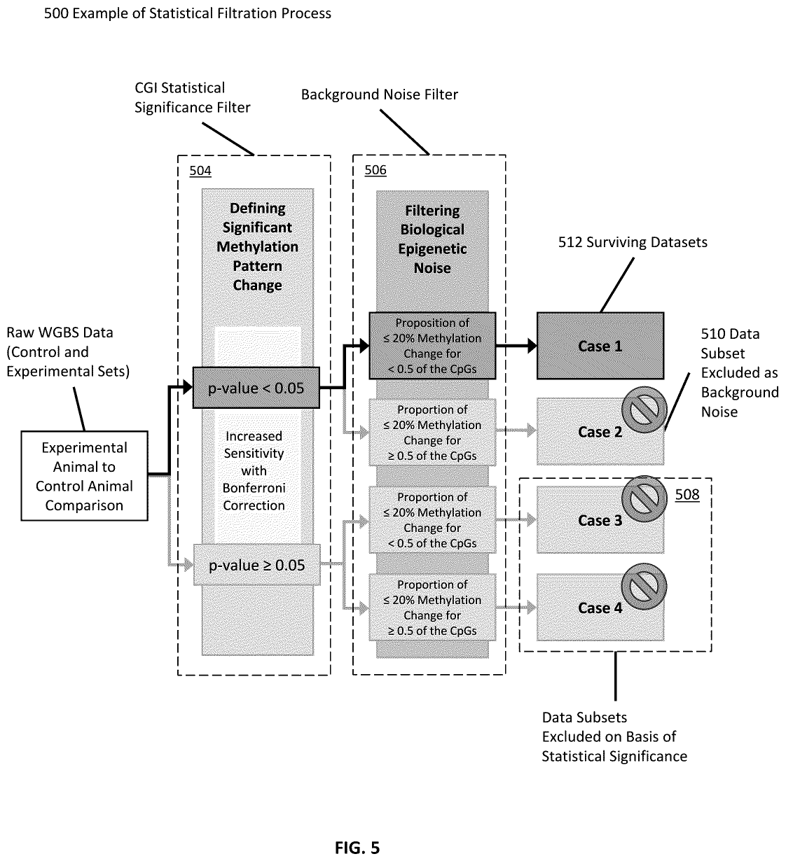

FIG. 5 depicts an example of a statistical-filter process 500 to categorize and rank CpG methylation patterns while filtering out datapoints that fail to meet thresholds for significance via a two-step evaluation approach. In some embodiments, a statistical-filter process may complement the LSTM autoencoder by filtering out insignificant data prior to step 104 of FIG. 1, thereby improving the overall efficiency of the method. In addition, the statistical-filter process may be used to verify training accuracy in process 200 or identify and screen for sequence data that contains CRISPR edits prior to step 202.

Specifically, in FIG. 5, a statistical filter is applied to raw WGBS data and uses non-computational detection algorithms to isolate CGI-regions and/or CpG sites by testing for significance among the dataset (using t-tests or p-value testing), or excluding data failing to meet certain thresholds (such as for variance or read depth) or lacking compelling genomic features that would support the likelihood a CRISPR-edit existing (such as proximity to PAM sites). The statistical filter produces filtered data, which is more tractable compared to unfiltered data and enriched for regions of interest.

In a first step, a first filter 504 calculates p-values for each CGI range within the given control and/or CRISPR-edited comparison using a Bonferroni correction. The filter then discards all of the CGIs 508 with insufficient p-values, resulting in a vastly reduced subset of viable experimental CGIs 510 and 512 that display significant epigenetic change relative to the control data.

In a second step, a second filter 506 reduces the remaining set of CGIs further through the application of an observed biological noise filter (accounting for minor methylation changes that could be part of normal cellular processes or introduced experimental biases). In this nonlimiting example, surviving CGIs are enriched for those displaying methylation changes beyond the determined 20% threshold for change. The sites edited using donor DNA and homology-directed repair mechanisms for genomic incorporation (i.e., CRISPR technology) display significant methylation changes and constitute the 512 surviving dataset. Filtered data 512 is converted into trainable data types before being passed to the neural network for processing.

Alternative embodiments of this statistical filtration approach may incorporate a statistical signal processing methodology that utilizes automated software to model the probability distribution of epigenetic noise data within a predefined range (i.e., evaluation window). Starting from a particular genomic location, the evaluation window slides and/or advances down a methylome of interest one genomic position at a time and compares the average amount of methylation between the unknown and multi-animal control sequences. Regions that surpass a particular standard deviation threshold are flagged while other data is filtered out. In accordance with some embodiments, this signal processing approach may be applied at the CGI and/or sub-CGI level.

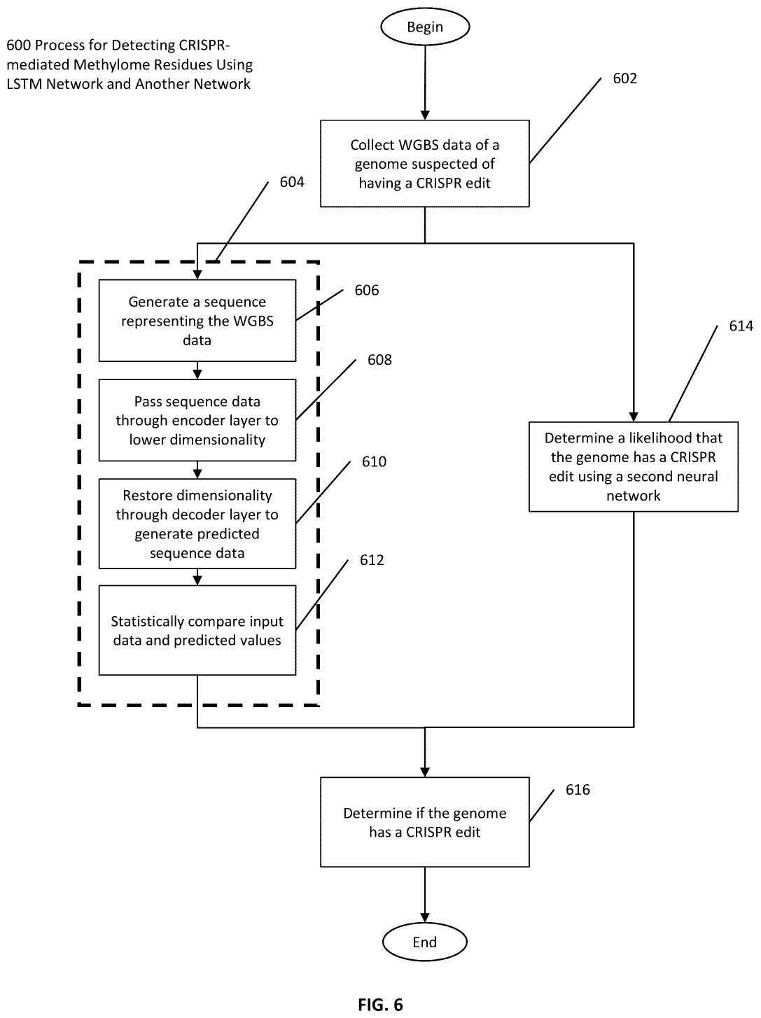

FIG. 6 is a flow diagram of another process 600 for detecting CRISPR-mediated methylome residues using a combination of steps 604 that utilizes a trained LSTM network 604 and a second step 614 that utilizes another neural network, in accordance with some embodiments.

The detection process 600 is capable of sequencing, filtering, learning, and interpreting multiple data streams before reaching an ultimate decision as to the location(s) of CRISPR-mediated residues within an epigenetic sample. Process 600 leverages both statistical analysis and multiple types of neural networks. In the example of FIG. 6, the ensemble includes both an LSTM autoencoder (incorporated within the combination of steps 604) and a convolution neural network (CNN) (i.e., computer vision-based approach), which is utilized in step 614.

The ensemble of neural networks is described herein as software, however the present disclosure is not limited in this regard, as other configurations are contemplated including where the ensemble is operated as a combination of software and hardware or purely as hardware.

In some embodiments, a CNN may be selected for its ability to rapidly extract features within visual representations (e.g., graphical plots, mappings, etc.) of methylation sequences without supervision. Supervised machine learning incorporates the use of pre-labeled examples as datasets to train algorithms to classify data. Because the identity of the input is already known, the aim of networks under a supervised learning approach is to accurately map the input data to its correct classification. By comparison, unsupervised machine learning directs a neural network to search for patterns in a dataset without labels, forcing the network to group the unsorted information according to similarities and differences it detects and then extract key features based on any underlying structure the network managed to identify within the dataset.

CNN models complement the LSTM approach—which often require supervision and longer training periods—and together they facilitate a more balanced treatment and/or evaluation of data. However, in some embodiments, it may be preferable to train the CNN module in a supervised fashion with feature-labeled training images in order to more accurately diagnose patterns as indicating CRISPR residues.

Similarly, in some embodiments, the LSTM network may be trained using supervised or unsupervised modalities, or by using a transfer-learning process, or by using some combination thereof, so as to optimize the overall compatibility and effectiveness with the CNN utilized in step 614. In some embodiments, both networks may be trained first in an unsupervised fashion and subsequently fine-tuned in a supervised fashion.

In attempting to screen and interpret input data as complex as that of the methylome, an ensemble of neural networks (as illustrated by FIG. 6) is preferable to any single neural network. Single neural networks are vulnerable to overfitting, underfitting, vanishing gradients, and achieving suboptimal error minimization. By comparison, a holistic, multi-network approach is better equipped to overcome problems related to bias and variance and achieve a more optimal result. The collaborative ensemble of networks may be connected and organized in a variety of ways. In some embodiments, multiple neural networks may be configured to operate in parallel, series, or within some higher-order network architecture depending on the optimal arrangement for the process.

In step 602, raw WGBS data containing a potential CRISPR-edit is collected, copied, assigned to a network, and then converted into a form compatible with that network. If the network is a CNN, visualizations of the data in the form of graphs and/or plots may be generated and utilized as inputs for the network. In step 606, discrete sequences are produced from the WGBS data. Once processed, each dataset is presented to its respective network. The datasets output by step 602 feed into the LSTM autoencoder 604 and the alternative network model employed in a step 614. In this example, the networks have been organized to operate concurrently.

The LSTM network detection process enclosed within 604 is highly similar to that of flowchart 100 in FIG. 1. Following the generation of input sequences in step 606, the data is passed into an encoder layer to lower dimensionality in a step 608. Following this, in a step 610, the dimensionality of the data is restored via a decoder layer. In a step 612, predicted values are compared to their original counterparts using statistical tests to identify locations that deviate from normal methylation patterns (i.e., potential sites of CRISPR-scarring). As this series of steps is executed, step 614 is performed in tandem using the second neural network, which generates its own set of determinations and/or predictions as to the location(s) of CRISPR-scars.

Within a data aggregation event occurring after steps 612 and 614 but prior to the final determination made in a step 616, the refined, LSTM autoencoder-derived CRISPR-scar data and the refined, CNN-derived CRISPR-scar data may be integrated to impute a final set of CRISPR-scar detections. FIG. 6 illustrates an example in which the features extracted by CNN and LSTM methods—and any other final parameterizations, biological factors, and/or evidence relevant to the given sample—are weighed according to their relative statistical strength and then combined by final data aggregator to detect final CRISPR-residues candidates. This ultimate synthesis can be performed as an extension of the neural network itself whereby one or more dataspaces are combined through regularization and/or dimensionality reduction.

Leveraging the outputs from both the LSTM module and CNN module may ultimately integrate methylation-sequence reconstruction, image processing and/or analysis, and feature extraction to advantageously screen for and/or diagnose CRISPR-scarring based on raw WGBS data, statistical correction, genomic regional restrictions, PAM site locations, or combinations thereof. Specifically, a final data aggregator may receive data from other sources, in addition to the outputs from the ANNs. In some embodiments, these data may include a plurality of alternative considerations including pathological results, clinical reports, laboratory tests, expanded genetic profiling, proteomic assays, etc., or combinations thereof.

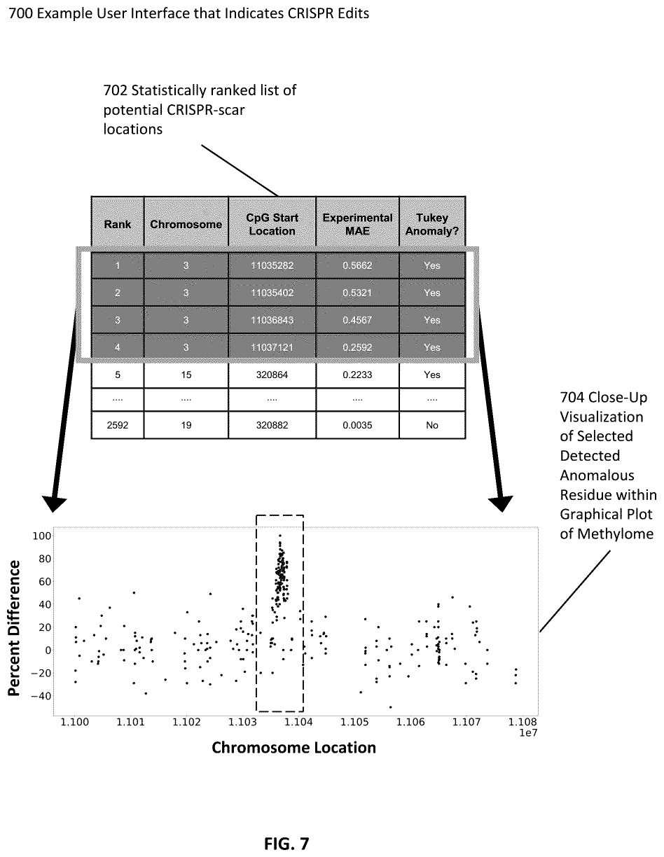

FIG. 7 illustrates an example user interface for indicating that a genome contains one or more methylation regions that has been CRISPR edited. As shown in FIG. 7, a table 702 conveys the exact locations of the detections by the model within a given sample as well as statistically ranks the likelihood that the detected areas contain CRISPR-scar sites. These regions of interest can also be conveyed visually in the form of a graphical plot of the methylome 704, which may be magnified to provide greater resolution and/or detail of an anomalous residue.

While illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive.

Furthermore, the steps of the disclosed routines may be modified in any manner, including by reordering steps and/or inserting or deleting steps. It is intended, therefore, that the specification and examples be considered as illustrative only, with a true scope and spirit being indicated by the following claims and their full scope of equivalents.

We claim:

1. A system for detecting a CRISPR-edited genome, comprising:
one or more processors configured to:
receive an input sequence data of a genome;
provide the input sequence data to a long short-term memory (LSTM) autoencoder neural network (ANN) having at least one encoder layer and at least one decoder layer, wherein the LSTM ANN was trained using a training data sequence of a genome without CRISPR edits;
reduce, using the encoder layer of the LSTM-ANN, a dimensionality of the input sequence data to generate reduced data;
restore, using the decoder layer of the LSTM-ANN, a dimensionality of the reduced data to generate restored data;
statistically compare the input sequence data and the restored data to identify anomalies in the genome; and
determine, based on a result of the statistical comparison, whether the genome contains a CRISPR-edited methylation region.

2. The system of claim 1, wherein the sequenced data is whole-genome bisulfite sequencing (WGBS) data.

3. The system of claim 1, wherein the input sequence data comprises of CpG start location data and methylation percentage data.

4. The system of claim 1, wherein one or more processors are further configured to determine whether, based on the identified anomaly in the genome, associated PAM sites have been disrupted and wherein the determination of whether the genome contains a CRISPR-edit is further based on the previous step.

5. The system of claim 1, wherein the statistically comparing includes performing a Tukey test on the input sequence data and the restored data.

6. The system of claim 1, wherein the determining includes determining a methylation location of the CRISPR edit.

7. The system of claim 1, wherein one or more processors are further configured to determine, using an Convoluted Neural Network (CNN), whether the genome likely contains a CRISPR-edited methylation site, wherein the determining includes weighing the generated score and results from the CNN to determine whether the genome contains a CRISPR edit.

8. A method for detecting a CRISPR-edited genome, comprising:
receiving input sequence data of a genome;
providing the input sequence data to an LSTM ANN having at least one encoder layer and at least one decoder layer, wherein the LSTM ANN was trained using a training data sequence of a genome without CRISPR edits;
reducing, using the encoder layer, a dimensionality of the input sequence data to generate reduced data;
restoring, using the decoder layer, a dimensionality of the reduced data to generate restored data;
statistically comparing the input sequence data and the restored data to identify anomalies in the genome; and
determining, based on a result of the statistical comparison, whether the genome contains a CRISPR-edited methylation region.

9. The method of claim 8, wherein the sequenced data is WGBS data.

10. The method of claim 8, wherein the input sequence data comprises of CpG start location data and methylation percentage data.

11. The method of claim 8, wherein determining includes determining whether, based on the identified anomaly in the genome, associated PAM sites have been disrupted and wherein determining of whether the genome contains a CRISPR-edit is further based on the previous step.

12. The method of claim 8, wherein the statistically comparing includes performing a Tukey test on the input sequence data and the restored data.

13. The method of claim 8, wherein the determining includes determining a methylation location of the CRISPR edit.

14. The method of claim 8, further comprising determining, using a CNN, whether the genome likely contains a CRISPR-edited methylation site, wherein the determining includes weighing the generated score and results from the CNN to determine whether the genome contains a CRISPR edit.

15. A non-transitory computer-readable storage medium storing instructions that when executed by a computer causes the computer to perform a method for detecting a CRISPR-edited genome, the method comprising:
receiving input sequence data of a genome;
providing the input sequence data to an LSTM ANN having at least one encoder layer and at least one decoder layer, wherein the LSTM ANN was trained using a training data sequence of a genome without CRISPR edits;
reducing, using the encoder layer, a dimensionality of the input sequence data to generate reduced data;
restoring, using the decoder layer, a dimensionality of the reduced data to generate restored data;
statistically comparing the input sequence data and the restored data to identify anomalies in the genome; and
determining, based on a result of the statistical comparison, whether the genome contains a CRISPR-edited methylation region.

16. The non-transitory computer-readable storage medium according to claim 15, wherein the sequenced data is WGBS data.

17. The non-transitory computer-readable storage medium according to claim 15, wherein the input sequence data comprises of CpG start location data and methylation percentage data.

18. The non-transitory computer-readable storage medium according to claim 15, wherein determining includes determining whether, based on the identified anomaly in the genome, associated PAM sites have been disrupted and wherein determining of whether the genome contains a CRISPR-edit is further based on the previous step.

19. The non-transitory computer-readable storage medium according to claim 15, wherein the determining includes determining a methylation location of the CRISPR edit.

20. The non-transitory computer-readable storage medium according to claim 15, further comprising determining, using a CNN, whether the genome likely contains a CRISPR-edited methylation site, wherein the determining includes weighing the generated score and results from the CNN to determine whether the genome contains a CRISPR edit.
